(19) United States
(12) Patent Application Publication
(10) Pub. No.: US 2023/0091716 A1
(43) Pub. Date: Mar. 23, 2023

(54) Progesterone Combinations

(71) Applicant: Glia, LLC, Boston, MA (US)

(72) Inventors: Zhonghui Katie LUO, Boston, MA (US); Kenneth I. SAWYER, Boston, MA (US); Wei-Wei CHANG, Boston, MA (US)

(21) Appl. No.: 17/904,035

(22) PCT Filed: Feb. 11, 2021
(86) PCT No.: PCT/US2021/017553

§ 371 Date: Aug. 11, 2022

Related U.S. Application Data
(60) Provisional application No. 62/975,640, filed on Feb. 12, 2020.

(51) Int. Cl. A61K 31/57; A61K 9/00; A61K 31/573; A61K 31/569; A61K 9/06; A61P 27/02; A61P 37/06; A61F 9/00

(52) U.S. Cl. CPC A61K 31/57; A61K 9/0014; A61K 9/0048; A61K 31/573; A61K 31/569; A61K 9/06; A61P 27/02; A61P 37/06; A61F 9/0017

(57) ABSTRACT

The present disclosure describes a method for treating or preventing an inflammatory ocular surface disease or disorder, such as ocular graft-versus-host disease, dry eye, meibomian gland disease, thyroid eye disease, blepharitis, Sjogren's syndrome, peripheral ulcerative keratitis, or Stevens-Johnson syndrome, in a human subject in need thereof, the method comprising (a) administering a topical progesterone composition to the forehead of the subject; (b) administering punctal plugs to the subject; and (c) administering a opical corticosteroid composition, such as loteprednol etabonate ointment, to the eye of the subject. Such administration methods may also be useful in improving corneal health before cataract surgery and/or promoting healing of the cornea after cataract surgery.

### CROSS-REFERENCES TO RELATED APPLICATIONS

This application claims the benefit of priority to U.S. Provisional Patent Application No. 62/975,640, filed on Feb. 12, 2020, the entire contents of which is herein incorporated by reference.

### BACKGROUND

Transdermal methods allow for the delivery of medicine directly through the skin. Gels, emulsions, creams, sprays and patches are easy to use and are effective for transdermal delivery of a drug. However, current transdermal delivery routes are utilized for delivering a drug either to exert a local effect or to enter the blood circulation.

Topical progesterone ointments are available commercially for use in hormone replacement therapy. However, the recommended daily dose of progesterone can lead to dose-dependent safety concerns.

Ocular graft-versus-host disease (GVHD) occurs in subjects who have undergone allogenic hematological stem cell transplantation. It can occur in subjects who have acute or chronic GVHD. Approximately 40-90% of subjects with chronic GVHD will develop ocular symptoms. Ocular manifestations can include keratoconjunctivitis sicca which may include notable blurred vision and photosensitivity, blepharitis (e.g., lid edema, lid ulceration, and/or lid erythema), corneal ulceration, corneal melt, corneal perforation, conjunctivial tissue damage, or neovascularization. Such symptoms can be moderate to severe. Often, ocular symptoms do not improve with treatment of the systemic GVHD.

Currently available ophthalmic solutions and suspensions, drugs, and devices largely have not been effective in alleviating the suffering of chronic ocular GVHD subjects. Accordingly, the need exists for new administration regimens for the effective treatment of inflammatory ocular surface diseases or disorders, such as ocular graft-versus-host disease.

### BRIEF SUMMARY

Provided herein is a method for treating or preventing an inflammatory ocular disease or condition in a human subject in need thereof, the method comprising: (a) administering a topical progesterone composition to the forehead of the subject; (b) administering punctal plugs to the subject; and (c) administering a topical corticosteroid composition to the eye of the subject.

Also provided herein is method for improving corneal health before cataract surgery and/or promoting healing of the cornea after cataract surgery in a human subject in need thereof, the method comprising: (a) administering a topical progesterone composition to the forehead of the subject; (b) administering punctal plugs to the subject; and (c) administering a topical corticosteroid composition to the eye of the subject.

Further provided is a method for treating ocular graft-versus-host disease in a human subject in need thereof, the method comprising: (a) administering a topical progesterone composition to the forehead of the subject; (b) administering punctal plugs to the subject; and (c) administering a topical loteprednol etabonate ointment composition to the eye of the subject.

Additionally, uses for the manufacture of a medicament and compositions for uses corresponding to the above methods are described.

### DETAILED DESCRIPTION

### I. General

The compositions and methods disclosed herein are directed to the combination of (a) topical administration of a progesterone composition to the forehead of a human subject, (b) punctal plugs in the eye of the subject, and (c) topical administration of a corticosteroid composition to the eye of the subject, to treat or prevent an inflammatory ocular surface disease or condition. Administering the combination of two drugs and one device results in a healthier ocular surface, which is useful in a number of methods, from treating or preventing an inflammatory ocular disease or condition to preconditioning the ocular surface, e.g., for other treatment, for example, surgery. For example, a method of treating ocular graft-versus-host disease in a human subject can include administering: (a) a topical progesterone composition to the forehead of a human subject, (b) punctal plugs to the eye of the subject, and (c) a topical corticosteroid composition, such as loteprednol etabonate ointment, to the eye of the subject.

A cranial drug administration route is disclosed wherein progesterone is topically administered. This route of administration is believed to innervate cranial nerves, e.g., the trigeminal nerves, providing a complementary modality for treatment of diseases and conditions that cannot be treated easily via the vascular system. The topical composition can contain progesterone in concentrations from about 0.01% by weight to about 20% by weight.

The progesterone can be formulated with appropriate excipients known in the art. The composition can be, e.g., a liquid or semi-solid, a solution, a suspension, an emulsion, a gel, a cream, a lotion, an ointment, or a patch. Administration can be simple or actively assisted by an electric current or other electrophysical device. The progesterone composition can be administered by applying it to the forehead.

Not to be bound by theory, it is believed that topical application of progesterone to the forehead and/or temple areas can result in rapid action via a cranial nerve, such as cranial nerve V (trigeminal nerve), VII (facial nerve), I, II, III, IV, VI, VIII, IX, X, XI, and/or XII. It is also believed that the rapid action of drugs administered in such a way can be attributed to drug absorption of the drug through the skin of the forehead, including absorption via the transappendageal route through hair follicles and/or sebaceous glands on the skin, uptake by receptors residing in nerve endings in the skin, and induction of signaling in the brain. The brain or a region of the brain, such as the hypothalamus, can then respond to the drug by sending appropriate signals to target muscles, glands, and organs. For example, a drug that affects a cranial nerve can exert a therapeutic effect, e.g., by subsequent downstream signaling on the hypothalamus-pituitary-gonadal (HPG) or hypothalamus-pituitary-adrenal (HPA) axis, on an organ or gland that is innervated by that nerve. Additionally, a lower dose of progesterone, as compared to the dose required for systemic delivery, can be effective, thereby enhancing safety.

### II. Definitions

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Prevent” or “prevention” or “preventing” as used herein refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to a subject in the absence of detectable inflammatory ocular disease or disorder (e.g., ocular graft-versus-host disease or dry eye) in the subject). The subject may be an individual at risk of developing the disease or disorder, such as an individual who has one or more risk factors known to be associated with development or onset of the disease or disorder. Thus, in certain embodiments, the term “preventing dry eye” refers to administering to a subject who does not have a detectable dry eye an anti-dry eye therapeutic substance. It is understood that the subject for anti-dry eye preventative therapy may be an individual at risk of developing anti-dry eye. It is also understood that prevention does not require a 100% success rate. In some instances, prevention may be understood as a reduction of the risk of dry eye, but not a complete elimination of the occurrence of dry eye.

### III. Compounds

The present disclosure is directed to topical compositions of progesterone, also known as pregn-4-ene-3,20-one, (8R,9S,10R,13S,14S,17S)-17-acetyl-10,13-dimethyl-1,2,6,7,8,9,11,12,14,15,16,17-dodecahydrocyclopenta[α]phenanthren-3-one, and CAS ID: 57-83-0, having the following chemical structure:

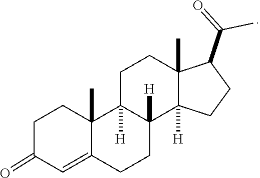

The present disclosure also contemplates topical compositions of a precursor of progesterone, including but not limited to pregnenolone.

Topical compositions of a corticosteroid are also described herein. In some embodiments, the corticosteroid comprises a low strength to medium strength corticosteroid. In some embodiments, the corticosteroid comprises loteprednol etabonate, betamethasone, dexamethasone, difluprednate, prednisolone, triamcinolone, rimexolone, or fluorometholone. Loteprednol etabonate, also known as 11β,17α-dihydroxy-21-oxa-21-chloromethylpregna-1,4-diene-3,20-dione 17α-ethylcarbonate and CAS ID: 82034-46-6, has the following chemical structure:

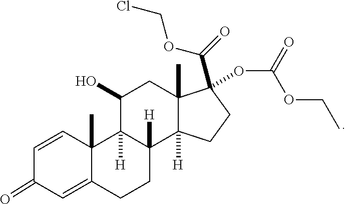

### IV. Compositions

In certain embodiments, the present disclosure provides a topical composition comprising progesterone and a pharmaceutically acceptable excipient. In some embodiments, the composition is a liquid, a semi-solid, a solution, a suspension, an emulsion, a gel, a cream, a lotion, or an ointment. In some embodiments, the composition is formulated in a gel or an ointment. In some embodiments, the composition is formulated for delivery in a patch. In some embodiments, the composition is formulated for topical or subcutaneous delivery in an implant device.

In some embodiments, a topical composition comprises progesterone in concentrations from about 0.001% to about 20%, e.g., from about 0.001% to about 10%, from about 0.005% to about 10%, from about 0.01% to about 10%, from about 0.01% to about 5%, from about 0.01% to about 2.5%, from about 0.1% to about 5%, or from about 0.1% to about 2.5%, by weight. For example, the progesterone concentration can be 0.001%, 0.025%, 0.005%, 0.01%, 0.025%, 0.05%, 0.1%, 0.2%, 0.25%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, 5%, 10%, or 20%, or any range defined by two numbers of the foregoing. In some embodiments, the topical composition comprises about 1% progesterone.

Topical compositions of a corticosteroid to the eye are provided in the methods described herein. When the corticosteroid is used in long-term administration regimens, the corticosteroid should be selected to minimize potential side effects, such as an increase in intraocular pressure (TOP). In some embodiments, the corticosteroid comprises a low to medium strength corticosteroid. In some embodiments, the corticosteroid comprises loteprednol etabonate, betamethasone, dexamethasone, difluprednate, prednisolone, triamcinolone, rimexolone, or fluorometholone. In some embodiments, the corticosteroid is loteprednol etabonate.

In some embodiments, a topical composition comprises a corticosteroid in concentrations from about 0.001% to about 20%, e.g., from about 0.001% to about 10%, from about 0.005% to about 10%, from about 0.01% to about 10%, from about 0.01% to about 5%, from about 0.01% to about 2.5%, from about 0.05% to about 1%, from about 0.1% to about 5%, or from about 0.1% to about 2.5%, by weight. For example, the corticosteroid concentration can be 0.001%, 0.025%, 0.005%, 0.01%, 0.025%, 0.05%, 0.1%, 0.2%, 0.25%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.5%, 5%, 10%, or 20%, or any range defined by two numbers of the foregoing. In some embodiments, the topical composition comprises about 0.5% corticosteroid.

The topical composition of corticosteroid loteprednol etabonate ointment used in the Examples described herein is commercially available. Lotemax™ (Bausch & Lomb) is a 0.5% (w/w) loteprednol etabonate ophthalmic ointment indicated for the treatment of postoperative inflammation and pain following ocular surgery.

### V. Administration

Progesterone may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the subject's life.

The dosage or dosing frequency of progesterone may be adjusted over the course of the treatment, based on the judgment of the administering physician.

The progesterone composition may be administered to a subject (e.g., a human subject) in an amount effective to produce the desired therapeutic effect. In certain embodiments, the composition is administered twice daily or once daily.

The progesterone composition can be administered topically to the face to the regions that are outside of the palpebral part of the eye. The palpebral part of the eye refers to the region of and around the eye associated with the palpebral component of the orbicularis oculi muscle group. The palpebral component of the muscles originates in the palpebral ligament and runs above and below the eye to the lateral angle of the eye, forming concentric circles around the eye. The palpebral part of the eye thus refers to the facial surface around the eye that corresponds to the location of the palpebral component of the orbicularis oculi muscle lying underneath the facial skin.

Non-limiting examples of facial regions for topical administration of progesterone include, for example, the forehead above the eyebrows, the temple area between the end of the eyebrow and the hairline including the temple region, the upper cheek, or the sides or bridge of the nose. In some embodiments, the progesterone composition is administered to the forehead. In some embodiments, the progesterone composition is administered to one or both temple regions. In some embodiments, the progesterone composition is administered to the upper cheek. In some embodiments, the progesterone composition is administered to one or both sides or the bridge of the nose. In some embodiments, the progesterone composition is administered to two or more regions of the face simultaneously or sequentially, and proximately or distant in time. For example, the progesterone composition can be administered to the forehead, and further administered to the temple region at the same time or at the next prescribed time, whether the next prescribed time is the same day or a different day. In some embodiments, the progesterone composition is administered to the same region of the face for each administration. In some embodiments, the progesterone composition is administered to any area of the skull, exclusive of the palpebral part of the eye.

In some embodiments, the amount of progesterone that is administered is from about 0.001 mg to about 20 mg, such as from 0.001 mg to 1 mg, from 0.001 mg to 0.8 mg, from 0.001 mg to 0.5 mg, from 0.001 mg to 0.25 mg, from 0.001 mg to 0.2 mg, from 0.01 mg to 1 mg, from 0.1 mg to 1 mg, from 0.01 mg to 0.5 mg, or from 0.01 mg to 0.25 mg. For example, the amount of progesterone that is administered can be about 0.001, 0.005, 0.01, 0.025, 0.05, 0.1, 0.2, 0.25, 0.3, 0.4, 0.5, 0.6, 0.7, 0.8, 0.9, 1, 2, 2.5, 5, 10, 20 mg, or any range defined by two numbers of the foregoing. In some embodiments, about 70 mg of a 1% progesterone composition, i.e., about 0.7 mg progesterone, is administered.

In some embodiments, the progesterone is administered in conjunction with at least one additional therapeutic agent. For instance, the topical progesterone composition can be administered in conjunction with punctal plugs and administration of a topical corticosteroid composition, e.g., loteprednol etabonate ointment, to the eye.

In some embodiments, the topical progesterone composition is administered for treatment of an inflammatory ocular disease or disorder, such as ocular GVHD. In some embodiments, the topical progesterone composition is administered in conjunction with administration of a topical corticosteroid composition, e.g., loteprednol etabonate ointment, to the eye of the subject. In some embodiments, the progesterone and the corticosteroid is administered sequentially, i.e., the progesterone is administered before the corticosteroid or the progesterone is administered after the administration of the corticosteroid. In some embodiments, the progesterone is administered about 5, 10, 15, 20, 30, 45, 60 minutes or more after the corticosteroid. In some embodiments, the corticosteroid is administered about 5, 10, 15, 20, 30, 45, 60 minutes or more after the progesterone. In some embodiments, the progesterone and the corticosteroid are administered simultaneously.

Punctal plugs, also known as punctum plugs, lacrimal plugs or occluders, are small, biocompatible devices that can be inserted into tear ducts to block drainage. This increases tear film and surface moisture in the eye to help relieve certain forms of dry eye. Two general types of tear duct plugs are: (i) semi-permanent, which can be made of long-lasting materials such as silicone, and (ii) dissolvable, which can be made of materials such as collagen that the body eventually absorbs. In some embodiments, the method comprises administering a progesterone composition in conjunction with punctal plugs.

The topical corticosteroid compositions used herein can be administered according to the directions of the individual approved indication for the composition. In some embodiments, the corticosteroid is administered to the eye of the subject. In some embodiments, the corticosteroid is administered as an eyedrop, e.g., formulated as a suspension. In some embodiments, the corticosteroid is administered by applying to the eyelids, e.g., the eyelid margins, and can be formulated as an ointment or a gel.

In some embodiments, the amount of corticosteroid that is administered is from about 0.001 mg to about 20 mg, such as from 0.001 mg to 1 mg, from 0.001 mg to 0.8 mg, from 0.001 mg to 0.5 mg, from 0.001 mg to 0.25 mg, from 0.001 mg to 0.2 mg, from 0.01 mg to 1 mg, from 0.1 mg to 1 mg, from 0.01 mg to 0.5 mg, or from 0.01 mg to 0.25 mg. For example, the amount of corticosteroid that is administered can be about 0.001, 0.005, 0.01, 0.025, 0.05, 0.1, 0.2, 0.25, 0.3, 0.4, 0.5, 0.6, 0.7, 0.8, 0.9, 1, 2, 2.5, 5, 10, 20 mg, or any range defined by two numbers of the foregoing. In some embodiments, the corticosteroid comprises a low to medium strength corticosteroid. In some embodiments, the corticosteroid comprises loteprednol etabonate, betamethasone, dexamethasone, difluprednate, prednisolone, triamcinolone, rimexolone, or fluorometholone. In some embodiments, the corticosteroid is loteprednol etabonate. In some embodiments, the corticosteroid composition is formulated as a gel or an ointment.

An administration regimen that can be useful to treat an ocular graft-versus-host disease in a human subject can include: (a) twice daily topical administration of 1% (w/w) progesterone composition to the forehead of the subject; and (b) once daily topical administration of 0.5% (w/w) loteprednol etabonate ointment to the eye, e.g., the eyelid margin, of the subject, in the presence of punctal plugs. When punctal plugs are not already present in the human, the method also includes administering punctal plugs to the human subject, e.g., such that all four puncti are plugged. In some embodiments, the progesterone composition is formulated in a gel.

### VI. Methods

The topical progesterone compositions described herein are generally useful in a method for treating or preventing an inflammatory ocular surface disease or disorder, such as ocular graft-versus-host disease (GVHD), dry eye, meibomian gland disease, thyroid eye disease, blepharitis, Sjogren's syndrome, peripheral ulcerative keratitis, or Stevens-Johnson syndrome, in a human subject in need thereof. In some embodiments, the inflammatory ocular surface disease or disorder is ocular GVHD, meibomian gland disease, thyroid eye disease, peripheral ulcerative keratitis, or Stevens-Johnson syndrome. In some embodiments, the inflammatory ocular surface disease or disorder is ocular GVHD. For example, a method for treating or preventing an inflammatory ocular disease or condition in a human subject in need thereof can include: (a) administering a topical progesterone composition to the forehead of the subject; (b) administering punctal plugs to the subject; and (c) administering a topical corticosteroid composition to the eye of the subject. When punctal plugs are already present in the human subject, a method for treating or preventing an inflammatory ocular disease or condition in a human subject in need thereof can include: (a) administering a topical progesterone composition to the forehead of the subject; and (b) administering a topical corticosteroid composition to the eye of the subject. In some embodiments, the progesterone composition is formulated in a gel. In some embodiments, the corticosteroid comprises a low to medium strength corticosteroid. In some embodiments, the corticosteroid comprises loteprednol etabonate, betamethasone, dexamethasone, difluprednate, prednisolone, triamcinolone, rimexolone, or fluorometholone. In some embodiments, the corticosteroid composition is formulated as a gel or an ointment. In some embodiments, the corticosteroid is loteprednol etabonate, e.g., loteprednol etabonate ointment. In some embodiments, the human subject is male. In some embodiments, the human subject is female. In some embodiments, the human subject is adult. In some embodiments, the human subject is pediatric.

In one specific application, a method for treating an ocular graft-versus-host disease in a human subject can include: (a) administering twice daily about 1% (w/w) progesterone composition to the forehead of the subject; and (b) administering once daily about 0.5% (w/w) loteprednol etabonate ointment to the eyes, e.g., the eyelid margins, of the subject, in the presence of punctal plugs. When punctal plugs are not already present in the human, the method also includes administering punctal plugs to the human subject, e.g., such that all four puncti are plugged. In some embodiments, the progesterone composition is formulated in a gel. The method can be performed in the subject for a desired period of time or duration, such as at least about one month, at least about 2 months, at least about 3 months, at least about 6 months, or at least about 12 months or longer. In one variation, the method is administered on a daily or intermittent schedule for the duration of the subject's life.

Ocular GVHD can occur in subjects who have undergone allogenic hematological stem cell transplantation. Ocular GVHD occurs when donor lymphocytes attack host histocompatibility antigens. It is believed to be a T-cell mediated process that leads to infiltration and inflammation of the lacrimal gland, conjunctiva, and ocular surface. The inflammation can eventually cause a decrease in the density of conjunctival goblet cells as well as scarring of the lacrimal gland and conjunctiva. Ocular GVHD is associated with meibomian gland obstruction, anterior and posterior blepharitis. There is often associated scarring of the lacrimal gland leading to decreased tear production. Conjunctival hyperemia with pseudomembrane and membrane formation can also be observed. Chronic inflammation can lead to conjunctival necrosis and cicatrical scarring and fibrosis. Subjects often have corneal manifestations including punctate epithelial keratopathy and filamentary keratitis. Severe disease can lead to corneal erosions, thinning, ulcerations, and possible perforations.

There are several diagnostic criteria for a clinician to use for assessing the severity of chronic ocular GVHD, including the NIH eye score, the Japanese dry eye score, and the DEWS 2007 score. See, e.g., Tatematsu, Y. et al. Sci. Rep. 2014; 4: 6680. The NIH eye score is a clinical scoring system proposed as NIH consensus criteria in 2005, as part of a global assessment of chronic GVHD severity based on the number of organs involved and the degree of impairment of the affected organs. The Japanese dry eye score, revised in 2006, is used in Japan for ocular GVHD as well as dry eye caused by other diseases. The dry eye workshop score (DEWS), reported in 2007, diagnoses dry eye based on dry eye symptomatology, tear film abnormality, conjunctival and corneal epithelial damage, and lid/meibomian gland dysfunction.

TABLE 1
NIH Eye Score

| Score | Description |
|---|---|
| 0 | Non dry eye |
| 1 | Mild dry eye |
| 2 | Moderate dry eye |
| 3 | Severe dry eye |

New ocular sicca documented by low Schirmer test values with a mean value of both eyes ≤5 mm at 5 minutes or a new onset of keratoconjunctivitis sicca by slit-lamp examination with mean values of 6 to 10 mm on the Schirmer test is sufficient for the diagnosis of chronic GHVD if accompanied by distinctive manifestations in at least 1 other organ.

TABLE 2
Japanese Dry Eye Score

| Score | Description |
|---|---|
| 0 | Non dry eye |
| 1 | Mild dry eye |
| 2 | Severe dry eye |

The Japanese dry eye criteria for diagnosis are: (1) disturbance of the tear film (Schirmer test ≤5 mm or tear film breakup time ≤5 seconds); (2) conjunctivocorneal epithelial damage (fluorescein staining score ≥3 points or rose bengal staining score ≥3 points); and (3) dry eye symptomatology. The presence of all three criteria is necessary for a diagnosis of definite dry eye disease. A score of 0 indicates non dry eye presenting no manifestations/symptoms, a score of 1 indicates symptoms, Schirmer test ≤5 mm, fluorescein score >3 points, and rose bengal score >3 points; and a score of 2 indicates symptoms, Schirmer test ≤5 mm, fluorescein score ≥3 points, and rose bengal score ≥3 points.

TABLE 3
DEWS 2007 Eye Score

| Score | Description |
|---|---|
| 0 | Non dry eye |
| 1 | Mild dry eye |
| 2 | Moderate dry eye |
| 3 | Severe dry eye |
| 4 | Very severe dry eye |

The DEWS 2007 score is determined from nine parameters, including symptoms, Schirmer test score, tear film breakup time, and abnormalities in the conjunctiva, cornea, tear, lid, and meibomian glands.

Other methods of assessing the degree and severity of the ocular GVHD include patient-reported questionnaires regarding the frequency and severity of dry eye symptoms, such as the Symptom Assessment iN Dry Eye (SANDE) or variations thereof. Other patient-reported data may include specific ocular surface disease symptoms questionnaires that include blurry vision, pain, and photosensitivity, e.g., the Ocular Surface Disease Index (OSDI), or variations thereof. See, e.g., Amparo F. et al. Ophthalmology 2015, 122(7):1498-503.

Dry eye is a condition in which a person does not have enough quality tears to lubricate and nourish the eye. Tears are necessary for maintaining the health of the front surface of the eye and for providing clear vision. Dry eye is a common and often chronic problem, particularly in older adults, and can vary from a mild to a severe condition.

Meibomian gland dysfunction (MGD) encompasses several meibomian gland disorders, ranging from congenital to acquired. Disruption of meibomian gland function negatively impacts both the quality and quantity of meibum secreted, which in turn affects ocular surface health through changes in tear film composition. Increased tear evaporation, hyperosmolarity, inflammation, and ocular surface damage can subsequently occur. This may cause discomfort, visual disruption, and sensation of dry eye.

Thyroid eye disease is a condition in which the eye muscles, eyelids, tear glands and fatty tissues behind the eye become inflamed. This can cause the eyes and eyelids to become red, swollen and uncomfortable and the eyes can be pushed forward (‘staring’ or ‘bulging’ eyes).

Blepharitis is inflammation of the eyelids. Blepharitis often can involve the part of the eyelid where the eyelashes grow and affects both eyelids. Blepharitis can occur when tiny oil glands located near the base of the eyelashes become clogged.

Sjogren's syndrome is a disorder of the immune system identified by its two most common symptoms—dry eyes and a dry mouth. The condition often accompanies other immune system disorders, such as rheumatoid arthritis and lupus. In Sjogren's syndrome, the mucous membranes and moisture-secreting glands of your eyes and mouth are often affected first—resulting in decreased tears and saliva.

Peripheral ulcerative keratitis (PUK), also known as peripheral corneal ulceration, is a potentially devastating disorder consisting of a crescent-shaped destructive inflammation at the margin of corneal stroma that is associated with an epithelial defect, presence of stromal inflammatory cells, and progressive stromal degradation and thinning. Commonly referred to as PUK, it can produce progressive necrosis of the corneal stroma, leading to perforation and blindness.

Stevens-Johnson Syndrome (SJS) is a disorder that causes painful blisters and lesions on the skin and mucous membranes and can cause severe eye problems. The most common cause of SJS is an adverse allergic drug reaction. Almost any drug can result in SJS, but sulfa drugs are a particularly common cause. It is more common in children and younger adults, but can develop at any age. Typical ocular problems associated with SJS can include conjunctivitis, scarring of the conjunctiva, inflammation inside the eye (iritis), corneal blisters and perforation, which can potentially lead to permanent vision loss.

The present standard of care for management of SJS is mainly supportive. The therapy can include ocular lubrication with artificial tears and ointments and frequent surveillance of ocular infections. Corneal transplants, limbal stem cell transplantations or artificial corneal procedures may be considered if advised by an ophthalmologist.

Because the compositions and methods described herein may be used to alleviate dry eye, they can also be used to facilitate other ophthalmic procedures, such as eye surgery. For example, the presently disclosed compositions and methods can be used to precondition an eye or portion of the eye, e.g., the cornea, prior to surgery, or help in healing of the eye or portion of the eye after surgery. In some embodiments, a method of improving corneal health before cataract surgery and/or promoting healing of the cornea after cataract surgery in a human subject in need thereof includes (a) administering a topical progesterone composition to the forehead of the subject; (b) administering punctal plugs to the subject; and (c) administering a topical corticosteroid composition to the eye of the subject. When punctal plugs are already present in the human subject, a method for improving corneal health before cataract surgery and/or promoting healing of the cornea after cataract surgery in a human subject in need thereof can include: (a) administering a topical progesterone composition to the forehead of the subject; and (b) administering a topical corticosteroid composition to the eye of the subject. In some embodiments, the progesterone composition is formulated in a gel. In some embodiments, the corticosteroid comprises a low to medium strength corticosteroid. In some embodiments, the corticosteroid composition is formulated as a gel or an ointment. In some embodiments, the corticosteroid comprises loteprednol etabonate, betamethasone, dexamethasone, difluprednate, prednisolone, triamcinolone, rimexolone, or fluorometholone. In some embodiments, the corticosteroid is loteprednol etabonate, e.g., loteprednol etabonate ointment.

Improving corneal health before cataract surgery can include facilitating accurate measurement prior to cataract surgery. Before a cataract surgery, a refraction can be performed to accurately determine the amount of nearsightedness, farsightedness and/or astigmatism. Additional measurements of the eyes can also be taken to determine the curvature of the cornea and the length of the eye.

Promoting healing of the cornea after cataract surgery can include reducing eye redness or reducing the time for the incision used during the surgery, e.g., a corneal incision, to heal.

Further provided herein is a topical progesterone composition for use in treating an inflammatory ocular surface disease or disorder, such as ocular graft-versus-host disease, dry eye, meibomian gland disease, thyroid eye disease, blepharitis, Sjogren's syndrome, peripheral ulcerative keratitis, or Stevens-Johnson syndrome, in a human subject in need thereof. In some embodiments, the inflammatory ocular surface disease or disorder is ocular GVHD. In some embodiments, a composition of topical progesterone can be for use in treating an inflammatory ocular surface disease or disorder in a human subject, wherein the composition is to be administered in conjunction with punctal plugs and a topical corticosteroid composition to the eye of the subject. In some embodiments, the corticosteroid composition is formulated as a gel or an ointment. For example, a composition of topical progesterone can be for use in treating ocular graft-versus-host disease in a human subject, wherein the composition is to be administered in conjunction with punctal plugs and topical corticosteroid loteprednol etabonate ointment to the eye of the subject. In some embodiments, the progesterone composition is formulated in a gel. In some embodiments, the human subject is male. In some embodiments, the human subject is female. In some embodiments, the human subject is adult. In some embodiments, the human subject is pediatric.

In some embodiments, a topical progesterone composition is for use in improving corneal health before cataract surgery and/or promoting healing of the cornea after cataract surgery in a human subject in need thereof. For example, a composition of topical progesterone can be for use in improving corneal health before cataract surgery and/or promoting healing of the cornea after cataract surgery in a human subject, wherein the composition is to be administered in conjunction with punctal plugs and topical corticosteroid loteprednol etabonate ointment to the eye of the subject. In some embodiments, the progesterone composition is formulated in a gel. In some embodiments, the human subject is male. In some embodiments, the human subject is female. In some embodiments, the human subject is adult. In some embodiments, the human subject is pediatric.

Also provided herein is the use of progesterone in the manufacture of a topical medicament for administration to a human subject in treating an inflammatory ocular surface disease or disorder, such as ocular graft-versus-host disease, dry eye, meibomian gland disease, thyroid eye disease, blepharitis, Sjogren's syndrome, peripheral ulcerative keratitis, or Stevens-Johnson syndrome. In some embodiments, the inflammatory ocular surface disease or disorder is ocular GVHD. In some embodiments, a topical progesterone medicament can be for treating an inflammatory ocular surface disease or disorder in a human subject and is administered in conjunction with punctal plugs and a topical corticosteroid composition to the eye of the subject. In some embodiments, the corticosteroid composition is formulated as a gel or an ointment. For example, a topical progesterone medicament can be for treating ocular graft-versus-host disease in a human subject and is administered in conjunction with punctal plugs and topical corticosteroid loteprednol etabonate ointment to the eye of the subject. In some embodiments, the progesterone composition is formulated in a gel. In some embodiments, the human subject is male. In some embodiments, the human subject is female. In some embodiments, the human subject is adult. In some embodiments, the human subject is pediatric.

In some embodiments, the use of progesterone in the manufacture of a topical medicament for administration to a human subject in improving corneal health before cataract surgery and/or promoting healing of the cornea after cataract surgery. In some embodiments, a topical progesterone medicament can be for improving corneal health before cataract surgery and/or promoting healing of the cornea after cataract surgery in a human subject and is administered in conjunction with punctal plugs and a topical corticosteroid composition to the eye of the subject. In some embodiments, the corticosteroid composition is formulated as a gel or an ointment. For example, a topical progesterone medicament can be for improving corneal health before cataract surgery and/or promoting healing of the cornea after cataract surgery in a human subject and is administered in conjunction with punctal plugs and topical corticosteroid loteprednol etabonate ointment to the eye of the subject. In some embodiments, the progesterone composition is formulated in a gel. In some embodiments, the human subject is male. In some embodiments, the human subject is female. In some embodiments, the human subject is adult. In some embodiments, the human subject is pediatric.

In some embodiments, the subject has one or more of the aforementioned diseases or conditions.

While the compositions and methods described herein are useful to treat a number of inflammatory ocular surface diseases or disorders, in extreme cases of inflammation, the subject would benefit from administration of at least one additional therapy.

The diagnosis and determination of treatment efficacy using a method described herein can be determined by a number of techniques known to a skilled artisan. For example, an ophthalmologist may evaluate a subject using a slit-lamp examination before, during, or after treatment.

### VII. Examples

Abbreviations. Certain abbreviations and acronyms are used in describing the experimental details. Although most of these would be understood by one skilled in the art, the following table contains a list of many of these abbreviations and acronyms.

| Abbreviation | Meaning |
|---|---|
| AML | acute myelocytic leukemia |
| IOP | intraocular pressure |
| LLL | Left lower lid |
| LUL | Left upper lid |
| OD | right eye |
| OS | left eye |
| OU | both eyes |
| RLL | Right lower lid |
| RUL | Right upper lid |
| PBSCT | peripheral blood stem cell transplant |
| PF-AT | preservative-free artificial tears/lubricant |
| SPK | Superficial punctate keratitis |

When used in the Examples, loteprednol etabonate ointment application consisted of a rice-grain sized amount of ointment applied with an eyeliner brush to each of the four lid margins (as if applying eyeliner on the waterline) unless otherwise indicated.

Pro-ocular™ (1% progesterone topical gel administered to the forehead twice daily) phase II trial for ocular GVHD included a randomized phase completed in September 2019, a cross-over phase completed in October 2019, and a subsequent open label phase.

All scoring is measured after fluorescein staining unless otherwise indicated.

Example 1. Case #1

In July 2017, a 67-year-old male subject with a history of AML treated with allogeneic PBSCT from a matched unrelated donor in October 2015 was evaluated. His ocular history included a history of spontaneous retinal detachment treated with scleral buckle OS.

In July 2017, the subject reported prominent crusting in the mornings, frequent itchiness and discomfort in both eyes. All symptoms got worse every evening to the point the irritation became continuous. He had to use lubricant eye drops (preservative containing over-the-counter artificial tears) at least 12 times a day for short periods of relief. He believed his bilateral eye symptoms started shortly after a second cataract surgery performed April 2017. Per his wife's report, the subject had prominent redness in both eyelids and sometimes on the eyeballs in the evenings. A slit-lamp examination showed blepharitis and superior corneal staining consistent with keratcoconjunctivitis sicca secondary to ocular GVHD. On the same visit, four plugs were placed in all puncti and loteprednol etabonate ointment on lid margin nightly was started. He switched to frequent preservative-free artificial tears as instructed. Subject's symptoms and signs both improved on the follow up visit 3 months later. However, a new retinal detachment was found in OS incidentally.

The subject had a successful retina surgery and resumed care in January 2019. Subject's ocular surface worsened presumably from the surgery, a long course of post-op eye drops, as well as the loss of punctal plugs and discontinuation of loteprednol etabonate ointment. All lost plugs were replaced, and loteprednol etabonate ointment was restarted on the lid margins in January 2019. His symptoms were better than that on the initial encounter, but still very bothersome to him He had filaments periodically and had cornea staining upon examination and sometimes abrasion from confluent staining. He also had prominent lid erythema and irregularity.

In July 2019, the subject started Pro-ocular™ treatment. He also reported taking oral Prednisone (2 mg). By September 2019, the subject reported definitive improvement with OD feeling completely normal and OS better. Slit-lamp examination showed less corneal staining. However, OS still bothered him so he was using lubricant frequently. At this visit, he admitted to not applying loteprednol etabonate ointment. After further discussion, he restarted nightly loteprednol etabonate ointment to the lid margin.

By November 2019, when he was still on the stable dose of oral prednisone 2 mg per day, he reported both eyes were “doing great,” “very comfortable,” and “feel normal.” Slit-lamp examination showed little to no findings on the lids and the ocular surface. His eye care consisted of preservative-free lubricant application no more than 2-3 times a day, loteprednol etabonate ointment applied on the lid margin nightly, and forehead application of Pro-ocular™ twice a day. Intraocular pressure was 16 (OD) and 14 (OS) without any other eye drops other than the above mentioned. The measured best corrected visual acuity (BCVA) was 20/20-1 in both eyes.

Slit Lamp Exam Case #1: July 2017

| | Right Eye | Left Eye |
|---|---|---|
| Lids/Lashes | 2+ rosacae, lid margin | 2+ rosacae, lid margin |
| | irregularity, 2+ erythema | irregularity, 2+ erythema |
| Conjunctiva/ | Normal | conjunctival scarring after |
| Sclera | | buckle |
| Cornea | trace SPK inferior rim, 2+ | 1+ SPK superior half |
| | coarse SPK superiorly | |

Slit Lamp Exam Case #1: November 2019

| | Right Eye | Left Eye |
|---|---|---|
| Lids/Lashes | formfit in RUL and RLL, | LUL and LLL plugs in, |
| | mild lid margin | mild lid margin |
| | irregularities and trace | irregularities and trace |
| | ulceration | ulceration |
| Conjunctiva/ | Normal, no staining | conjunctival scarring |
| Sclera | | after buckle |
| Cornea | mucous in tear film, rare | mucous in tear film, no |
| | SPK | SPK |

### Example 2. Case #2

In September 2015, a 61-year-old male subject with a history of AML treated with PBSCT from a matched, related donor in 2011 was evaluated. He had severe chronic GVHD involving multiple organs and his eyes one year after the transplant. His systemic GVHD was well controlled and he was already off immunosuppression. However, his eyes remained symptomatic. He had treatment previously including bilateral lower punctal plugs that were still present, which he stated helped but never led to any significant improvement. He had mild ocular surface staining and mild blepharitis as well significant cataracts that affected his vision. He also had a self-reported ocular history of steroid responder and possible hepatitis simplex keratitis in the right eye.

In September 2015, both upper puncti were plugged. Cyclosporine ophthalmic emulsion (ReStasis™) and loteprednol etabonate ointment to lid margin were started as well as frequent preservative-free lubricant. The subject reported some improvement. Subsequently, successful cataract surgery was performed in both eyes in early 2016. By May 2016, subject reported that despite excellent vision, both eyes were burning, itchy, and teary with frequent foreign body sensation and photophobia. He was treated with ReStasis, loteprednol etabonate ointment, and preservative lubricant every 2 hours with all puncti plugged.

Subject was frustrated and stopped coming in for follow up until 2019 when he came in for the Pro-ocular™ phase II trial screening. His eyes were red and irritated with uncorrected vision 20/25 OD and 20/30 OS with both lower lid plugs in place. He was on loteprednol etabonate ointment intermittently and frequently used preservative-free lubricant. Slit-lamp examination showed mild yet persistent conjunctival injections and corneal staining in both eyes, as well as moderate edema, erythema and marginal ulceration of all lids.

In July 2019, the subject started the randomized phase II Pro-ocular™ trial. By September 2019 at the end of the randomized phase, he was surprised to find that he was in the active arm as he was not sure there was any significant improvement at all. Slit-lamp examination showed improvement in conjunctival injections but no improvement elsewhere. Upon questioning, the subject reported stopping loteprednol etabonate ointment since entering the trial. Therefore, loteprednol etabonate ointment treatment to lid margin was restarted.

Within 3 days, the subject reported that his eye symptoms were much improved (80-85% in his words). By December 2019, he reported OD was “90% better”, OS was improved “beyond what I thought was possible”. He did not need lubricant every day. The subject strongly stated that the combination of loteprednol etabonate ointment and Pro-ocular™ was better than any other treatment he had ever received. His uncorrected vision was 20/25+2 OD and 20/25 OS. His IOP was 12 and 13. His lids were in excellent condition and cornea staining minimal in both eyes.

Slit Lamp Exam Case #2: September 2015

| | Right Eye | Left Eye |
|---|---|---|
| Lids/Lashes | lower plug in, 1+ erythema | lower plug in, 1+ erythema |
| | 1+ edema at margin, 1+ | 1+ edema, 2+ lid margin |
| | lid margin ulceration | ulceration |
| Conjunctiva/ | 2+ superior limbal staining | 2+ superior limbal staining |
| Sclera | | |
| Cornea | 1+ scattered SPK | Coarse patches (2+) of SPK |
| | | inferiorly |

Slit Lamp Exam Case #2: December 2019

| | Right Eye | Left Eye |
|---|---|---|
| Lids/Lashes | lower plug in, upper | lower plug in, upper |
| | plug gone, no edema or | plug gone, no edema or |
| | erythema, trace lid | erythema, trace lid |
| | margin ulceration | margin ulceration |
| Conjunctiva/ | no superior staining, | trace superior limbal |
| Sclera | 1+ temporal and nasal | staining, trace staining |
| | staining | temporally |
| Cornea | no superior staining, | no superior staining, |
| | 0.5+ SPK inferior quarter | 0.5+ SPK inferior quarter |

A follow-up after ten additional months on progesterone/loteprednol etabonate/punctal plugs showed that the patient maintained improved eye condition. Uncorrected visual acuity 20/20 OD, 20/40 OS, IOP 10 and 12.

| | Right Eye | Left Eye |
|---|---|---|
| Lids/Lashes | Both plug ins. Medial | lower plug in, upper |
| | and lateral canthal area | plug gone, trace lid |
| | irritated. Rest of the lid | margin ulceration |
| | margins in good condition | |
| Conjunctiva/ | no superior staining, | No superior staining, |
| Sclera | half+ temporal staining | half+ staining temporally |
| Cornea | ½+ superior staining, | no superior staining, |
| | diffuse 1+ fine SPK rest | trace SPK at inferior |
| | of cornea. Copious amount | margin, minimal mucous |
| | of long string mucous | |

### Example 3. Case #3

In April 2018, a 32-year-old male subject with a history of AML treated with PBSCT from a matched sibling donor in April 2017 was evaluated. He had chronic skin GVHD treated with oral prednisone taper. In April 2017, he was only on tacrolimus 1 mg per day for immunosuppression.

Subject reported sudden onset ocular symptoms of foreign body sensation, pain and hazy vision upon tapering off oral prednisone. He was seen by a local ophthalmologist who started him on Tobradex® (tobramycin and dexamethasone ophthalmic suspension) four times a day and Muro 128 (2% hypertonic saline) eye drops in addition to frequent preservative-free lubricant. Despite reported improvement, slit-lamp examination showed severe keratoconjunctivitis sicca with confluent conjunctival and corneal staining that put him in high risk of abrasion or even corneal melt. His corrected vision was 20/30-1 OD and 20/25-1 OS.

All four puncti were immediately plugged. Tobradex® and Muro 128 were stopped due to surface toxicity. PF-AT was increased to every hour while awake. Loteprednol etabonate ointment (equivalent of an eye drop) was started at higher dose as a step-down to Tobradex®. Subject reported significant improvement in 4 weeks. However, there was persistent corneal staining, redness and irritation in both eyes during the following year. His corneal epithelium was fragile with frequent filaments despite frequent PF-AT use every 15 to 30 minutes. Of note, his lids never experienced significant inflammation. Punctal plugs often fell out and were replaced after loss.

He had some episodes of systemic ocular GVHD flare-up and was placed on oral prednisone (30 mg) in August 2018, which was tapered to 12.5 mg by April 2019 and maintained at 12.5 mg as of December 2019. His cataracts started to progress but surgery could not be planned as his cornea surface was in very poor condition that did not allow accurate measurement for lens power calculation. In addition, concerns of non-healing surgical wound further discouraged cataract surgery.

The subject entered the placebo arm in the Pro-ocular™ phase II trial while continuing loteprednol etabonate ointment daily treatment as well as frequent PF-AT. He reported no improvement and firmly stated “no change” during the trial visits. No significant improvement was found on slit-lamp examination either.

To better understand the role of each treatment component, a step-wise regimen was evaluated. In September 2019, the subject received Pro-ocular™ treatment in the crossover phase. In October 2019, he reported some improvement. Further, his colleagues commented his eyes were less red. One missing plug from each eye was replaced (they were lost during the trial and could not be replaced during the trial due to protocol restriction). In November 2019, OD appeared much better as both plugs were in place while OS was not as good with another missing plug. The missing plug was replaced and loteprednol etabonate ointment was added to OS lid margin only in both eyes. In December 2019, both eyes were in excellent condition that allowed accurate measurement. Cataract surgery was successfully performed on the right eye in December 2019 and on the left eye January 2020, followed by uneventful post-operative healing. Both eyes exhibited a corrected vision of 20/20 with the refractive aim right on target. His eyes looked so white and comfortable, that one of the technicians could not believe he was the same “poor guy on the trial” from a few months ago.

Slit Lamp Exam Case #3: April 2018

| | Right Eye | Left Eye |
|---|---|---|
| Lids/Lashes | Normal | Normal |
| Conjunctiva/ | 2+ staining nasally, 2+ | 2+ staining nasally, |
| Sclera | staining temporally | 1+ staining temporally |
| Cornea | 4+ confluent punctate | 2+ punctate SPK inferior half, |
| | fine staining of entire | 2+ staining superior half, |
| | cornea including superiorly | relatively less involvement |
| | | temporally and over pupil |

Slit Lamp Exam Case #3: January 2020

| | Right Eye | Left Eye |
|---|---|---|
| Lids/Lashes | both plugs in, no lid margin | both plugs in, no lid margin |
| | ulceration | ulceration |
| Conjunctiva/ | Normal, no staining | Normal, no staining |
| Sclera | | |
| Cornea | 0.5+ superior staining, few | 1+ diffuse faint SPK |
| | SPK inferiorly | |

A follow-up after ten additional months on progesterone/loteprednol etabonate/punctal plugs showed that the patient maintained improved eye condition. Uncorrected visual acuity 20/20 OD, 20/25 OS, IOP 12 and 11.

| | Right Eye | Left Eye |
|---|---|---|
| Lids/Lashes | both plugs in, no lid | upper plug in, lower |
| | margin ulceration, minimal | lost, no lid margin |
| | crusting on skin | ulceration |
| Conjunctiva/ | no staining, trace | 1+ staining superiorly and |
| Sclera | injection nasally. | nasally, trace temporally |
| | | and inferiorly |
| Cornea | very faint, few scattered | 1+ diffuse coarse SPK |
| | SPK | entire cornea, no specific |
| | | superior staining |

### Example 4. Case #4

In May 2018, a 65-year-old male subject with a history of AML treated with PBSCT from a matched unrelated donor in November 2017 was evaluated. He had very mild chronic GVHD involving his skin that required low dose immunosuppression of 1 mg tacrolimus daily, which was tapered off prior to June 2019.

The subject presented with bilateral lid laxity, redness, thickening, and unilateral keratoconjunctivitis sicca with corneal staining only prominent in the right eye. He reported itchiness and grittiness that was the worst in the morning. In May 2018, punctal plugs were only placed in the right eye with noticeable improvement, while 4 weeks later OS became symptomatic. In the following year, the cornea surface was fairly well maintained in both eyes as long the plugs stayed in. However, his conjunctival redness were always very pronounced in both eyes. He had been using PF-AT frequently, often hourly. Subject reported his eyes became extremely red after driving.

In July 2019, he participated in the active arm of the Pro-ocular™ phase II trial. The subject reported immediate improvement. In September 2019, he no longer needed the PF-AT first thing in the morning and the lids became less red and stiff. However, his “eyeballs” were still red and irritated especially after driving, and, therefore, still needed PF-AT 4-6 times a day. He lost one plug from each eye in the meantime. In September 2019, both plugs were replaced, and he was started on loteprednol etabonate ointment to the lid margins for the first time. By October 2019, both eyes had improved significantly. His eyeballs and eyelids were no longer red. He only used PF-AT once or twice a day in addition to the Pro-ocular™ forehead application and loteprednol etabonate ointment nightly application. He reported that his family members were very amazed by his transformation in the prior few months.

By January 2020, he had successful cataract surgeries in both eyes with smooth recovery. His uncorrected vision was 20/20 at 1 week after each surgery with normal IOP.

Slit Lamp Exam Case #4: May 2018

| | Right Eye | Left Eye |
|---|---|---|
| Lids/Lashes | 2.5 mm fluid containing cyst on | 2+ lid margin irregularity |
| | RL, 2+ lid margin irregularity | and erythema, lid laxity |
| | and erythema, lid laxity | |
| Conjunctiva/ | Normal | Normal |
| Sclera | | |
| Cornea | poor tear film, 2+ SPK inferior | Normal, no SPK |
| | quarter | |

Slit Lamp Exam Case #4: January 2020

| | Right Eye | Left Eye |
|---|---|---|
| Lids/Lashes | Lloppy lids, both plugs in, | floppy lids, both plugs in, |
| | trace foamy discharge, trace | trace foamy discharge, trace |
| | erythema, trace edema | erythema, trace edema |
| Conjunctiva/ | No conjunctiva staining or | No conjunctiva staining or |
| Sclera | injection | injection |
| Cornea | Normal, no staining | Normal, no staining |

A follow-up after nine additional months on progesterone/loteprednol etabonate/punctal plugs showed that the patient maintained improved eye condition. Uncorrected visual acuity 20/20+1 OD, 20/20+2 OS, IOP 10 and 10.

| | Right Eye | Left Eye |
|---|---|---|
| Lids/Lashes | both plugs in, floppy | both plugs in, floppy |
| | eyelids, trace margin | eyelids, 1+ margin |
| | erythema and ulceration. | erythema and ulceration. |
| | No edema, notch or | No edema, notch or |
| | irregularity | irregularity |
| Conjunctiva/ | no staining, no injection. | 1+ temporal staining, |
| Sclera | 1+ conjunctivochalasis | trace injection. 1+ |
| | | conjunctivochalasis |
| Cornea | No staining | Trace staining inferiorly, |
| | | no superior staining |

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method for treating or preventing an inflammatory ocular disease or condition in a human subject in need thereof, the method comprising:
(a) administering a topical progesterone composition to the forehead of the subject;
(b) administering punctal plugs to the subject; and
(c) administering a topical corticosteroid composition to the eye of the subject.

2. The method of claim 1, wherein the inflammatory ocular disease or condition comprises ocular graft-versus-host disease, dry eye, meibomian gland disease, thyroid eye disease, blepharitis, Sjogren's syndrome, peripheral ulcerative keratitis, or Stevens-Johnson syndrome.

3. The method of claim 1, wherein the inflammatory ocular disease or condition is ocular graft-versus-host disease.

4. A method for improving corneal health before cataract surgery and/or promoting healing of the cornea after cataract surgery in a human subject in need thereof, the method comprising:
(a) administering a topical progesterone composition to the forehead of the subject;
(b) administering punctal plugs to the subject; and
(c) administering a topical corticosteroid composition to the eye of the subject.

5. The method of any one of claims 1 to 4, wherein the corticosteroid comprises a low to medium strength corticosteroid.

6. The method of any one of claims 1 to 5, wherein the corticosteroid comprises loteprednol etabonate, betamethasone, dexamethasone, difluprednate, prednisolone, triamcinolone, rimexolone, or fluorometholone.

7. The method of any one of claims 1 to 6, wherein the corticosteroid is formulated in a gel or an ointment.

8. A method for treating ocular graft-versus-host disease in a human subject in need thereof, the method comprising:
(a) administering a topical progesterone composition to the forehead of the subject;
(b) administering punctal plugs to the subject; and
(c) administering a topical loteprednol etabonate ointment composition to the eye of the subject.

9. The method of any one of claims 1 to 8, wherein the progesterone composition comprises from 0.01% to 10% progesterone by weight.

10. The method of any one of claims 1 to 8, wherein the progesterone composition comprises from 0.1% to 5% progesterone by weight.

11. The method of any one of claims 8-10, comprising twice daily or once daily administration of the progesterone composition.

12. The method of any one of claims 8-11, comprising administering from 0.01 mg to 10 mg progesterone daily.

13. The method of any one of claims 1 to 12, wherein the corticosteroid composition comprises from 0.05% to 1% corticosteroid.

14. The method of any one of claims 1 to 13, comprising once daily administration of the corticosteroid composition.

15. The method of any one of claims 1 to 14, wherein the method further comprises administration of at least one additional therapy.

16. A method for treating ocular graft-versus-host disease in a human subject in need thereof, the method comprising:
(a) administering a topical 1% progesterone composition to the forehead of the subject twice daily;
(b) administering punctal plugs to the subject; and
(c) administering a topical 0.5% loteprednol etabonate ointment composition to the eye of the subject once daily.

17. Use of progesterone in the manufacture of a topical medicament for administration to the forehead of a human subject in treating or preventing an inflammatory ocular disease or condition in the subject, wherein the use further comprises administration of punctal plugs and a topical corticosteroid composition to the eye of the subject.

18. Use of progesterone in the manufacture of a topical medicament for administration to the forehead of a human subject in improving corneal health before cataract surgery and/or promoting healing of the cornea after cataract surgery in the subject, wherein the use further comprises administration of punctal plugs and a topical corticosteroid composition to the eye of the subject.

19. Use of progesterone in the manufacture of a topical medicament for administration to the forehead of a human subject in treating ocular graft-versus-host disease in the subject, wherein the use further comprises administration of punctal plugs and a topical corticosteroid loteprednol etabonate ointment composition to the eye of the subject.

20. A topical composition of progesterone for use in treating or preventing an inflammatory ocular disease or condition in a human subject, wherein the composition is to be administered in conjunction with punctal plugs and a topical corticosteroid composition to the eye of the subject.

21. A topical composition of progesterone for use in improving corneal health before cataract surgery and/or promoting healing of the cornea after cataract surgery in a human subject, wherein the composition is to be administered in conjunction with punctal plugs and a topical corticosteroid composition to the eye of the subject.

22. A topical composition of progesterone for use in treating ocular graft-versus-host disease in a human subject, wherein the composition is to be administered in conjunction with punctal plugs and a topical corticosteroid loteprednol etabonate ointment composition to the eye of the subject.
